(19) United States
(12) Patent Application Publication
(10) Pub. No.: US 2023/0138886 A1
(43) Pub. Date: May 4, 2023

(54) LEARNING DEVICE, PERISHABLE PRODUCT CONTAINING DEVICE, AND NON-TRANSITORY COMPUTER-READABLE STORAGE MEDIUM

(71) Applicant: DAIKIN INDUSTRIES, LTD., Osaka-shi, Osaka (JP)

(72) Inventors: Shouichi TANNO, Osaka-shi, Osaka (JP); Kiichirou SATOU, Osaka-shi, Osaka (JP); Hidenori MATSUI, Osaka-shi, Osaka (JP)

(73) Assignee: DAIKIN INDUSTRIES, LTD., Osaka-shi, Osaka (JP)

(21) Appl. No.: 17/915,660

(22) PCT Filed: Mar. 30, 2021
(86) PCT No.: PCT/JP2021/013482

§ 371 Date: Sep. 29, 2022

(30) Foreign Application Priority Data
Mar. 31, 2020 (JP) 2020-065240

(51) Int. Cl. G06N 20/00; G06F 18/21

(52) U.S. Cl. CPC G06N 20/00; G06F 18/217

(57) ABSTRACT

Reinforcement learning is performed on control conditions of the perishable product environment by using information regarding freshness of perishable products by a freshness sensor to automatically control the perishable product environment. There are provided: a freshness determination section 520 acquiring information regarding freshness of a perishable product contained in a storage container; and an analysis section 530 learning, by reinforcement learning, an inside environment of the storage container for the freshness of the perishable product acquired by the freshness determination section 520 to decide a reward used in the learning. The analysis section 530 decides the reward based on the decrease in the freshness over a certain period of time under the inside environment for the freshness determined based on the freshness acquired by the freshness determination section 520. Then, the analysis section 530 learns the inside environment for the freshness based on the decided reward.

### TECHNICAL FIELD

The present disclosure relates to a learning device, a perishable product containing device, and a program.

### BACKGROUND ART

Patent Document 1 discloses that the internal environment is controlled by inferring the amount of activated gas generated in time for the desired shipment timing and generating the activated gas based on the storage knowledge base, which is made by ruling of the optimum storage conditions based on the past storage experience and bringing thereof into knowledge, and environmental measurement information regarding gas metabolism amount. It is also described that software processing using regression analysis, which is a system of machine learning, is used to infer the amount of generated activated gas.

### CITATION LIST

### Patent Literature

Patent Document 1: Japanese Patent Laid-Open No. 2019-41601

### SUMMARY OF THE INVENTION

### Technical Problem

The freshness control of perishable products is, for example, different by each perishable product, or changed in accordance with initial freshness at the time when a perishable product is contained in the container. For this reason, though the environment is controlled under the same uniform control conditions, it is difficult to perform proper freshness control.

An object of the present disclosure is to perform reinforcement learning on control conditions of the perishable product environment by using information regarding freshness of perishable products obtained by a freshness sensor to automatically control the perishable product environment.

### Solution to Problem

A learning device of the present disclosure includes: a freshness information acquisition unit acquiring information regarding freshness of a perishable product contained in the storage container; a learning unit learning the inside environment of the storage container for the freshness of the perishable product acquired by the freshness information acquisition unit; and a reward decision unit deciding a reward used by the learning unit, wherein the reward decision unit decides the reward based on a decrease in the freshness over a certain period of time under the inside environment for the freshness determined based on the freshness acquired by the freshness information acquisition unit, and the learning unit learns the inside environment for the freshness based on the reward decided by the reward decision unit.

This makes it possible to perform reinforcement learning on control conditions of the perishable product environment by using the information regarding the freshness of the perishable product to automatically control the perishable product environment.

Here, when inside environments before and after the certain period of time are constant, the reward decision unit may decide the reward based on an inside environment at a start point of the certain period of time.

This makes it possible to learn the environmental control to provide the constant inside environment over the certain period of time.

In addition, when inside environments before and after the certain period of time are different, the reward decision unit may decide the reward based on an inside environment at a specific point during the certain period of time.

This makes it possible, in the case where the inside environment changes during the certain period of time, to learn the environmental control based on the inside environment at the specific point.

In addition, when inside environments before and after the certain period of time are different, the reward decision unit may decide the reward based on an inside environment at a start point and an inside environment at an end point of the certain period of time.

This makes it possible, in the case where the inside environment changes during the certain period of time, to learn the environmental control based on the inside environments at the start point and the end point of the certain period of time.

In addition, when inside environments before and after the certain period of time are different, the reward decision unit may find a representative value of environmental information values indicating inside environments at multiple points during the certain period of time, and may decide the reward based on the representative value.

This makes it possible to learn the environmental control in response to the change in the inside environment over a certain period of time.

Moreover, a containing device of the present disclosure is a perishable product containing device including: a storage container containing a perishable product; an adjustment unit adjusting an inside environment of the storage container, the inside environment including at least a temperature; an environmental information acquisition unit acquiring environmental information inside the container, the environmental information including at least the temperature; a freshness measurement unit measuring freshness of the perishable product contained in the storage container; a learning unit learning the inside environment of the storage container for the freshness of the perishable product measured by the freshness measurement unit; and a reward decision unit deciding a reward used by the learning unit, wherein, based on the freshness measured by the freshness measurement unit, the environmental information acquired by the environmental information acquisition unit, and a learning result of the learning unit, the adjustment unit operates to cause the inside environment of the storage container to serve as an inside environment for the freshness, the reward decision unit decides the reward based on a decrease in the freshness over a certain period of time under the inside environment adjusted by the adjustment unit, and the learning unit learns the inside environment for the freshness based on the reward decided by the reward decision unit.

This makes it possible to perform reinforcement learning on control conditions of the inside environment by using the information regarding the freshness of the perishable product obtained from the storage container to automatically control the perishable product environment.

Here, the learning unit may learn the inside environment for the freshness of the perishable product measured by the freshness measurement unit to maximize the reward decided by the reward decision unit, and the adjustment unit may adjust the inside environment of the storage container to cause the environmental information acquired by the environmental information acquisition unit to serve as the inside environment for the freshness learned by the learning unit.

This makes it possible to learn the control of the inside environment optimized with respect to the decrease in the freshness of the perishable product.

In addition, when inside environments before and after the certain period of time are different, the reward decision unit may decide the reward based on an inside environment at one or more points during the certain period of time.

This makes it possible to learn the environmental control in response to the change in the inside environment over a certain period of time.

In addition, the inside environment at one or more points during the certain period of time may include the inside environment at an end point of the certain period of time.

This makes it possible to learn the environmental control assuming the inside environment when the certain period of time has elapsed.

Moreover, the program of the present disclosure causes a computer to implement: a function of acquiring information regarding freshness of a perishable product contained in a storage container; a function of learning an inside environment of the storage container for the acquired freshness of the perishable product; and a function of deciding a reward used in the learning, wherein, in the function of deciding the reward, the reward is decided based on a decrease in the freshness over a certain period of time under the inside environment for the freshness, the inside environment being determined based on the freshness of the perishable product acquired by the function of acquiring the information regarding the freshness, and, in the function of learning, the inside environment for the freshness is learned based on the reward decided by the function of deciding the reward.

According to the computer installing the program, the reinforcement learning can be performed on the control conditions of the perishable product environment by using the information regarding the freshness of the perishable product to automatically control the perishable product environment.

### BRIEF DESCRIPTION OF DRAWINGS

FIG. 1 is a diagram showing an overall configuration of a perishable product control system to which the present embodiment is applied;

FIG. 2 is a diagram showing a functional configuration example of an information processing device;

FIG. 3 is a diagram showing a hardware configuration example of the information processing device;

FIG. 4 is a diagram showing an example of an inside environment (inside temperature) over a certain period of time adjusted by an environment adjustment device;

FIG. 5 is a diagram showing another example of the inside environment (inside temperature) over a certain period of time adjusted by the environment adjustment device;

FIG. 6 is a diagram showing still another example of the inside environment (inside temperature) over a certain period of time adjusted by the environment adjustment device; and

FIG. 7 is a diagram showing still another example of the inside environment (inside temperature) over a certain period of time adjusted by the environment adjustment device.

### DESCRIPTION OF EMBODIMENT

Hereinafter, an embodiment will be described in detail with reference to attached drawings.

### <System Configuration>

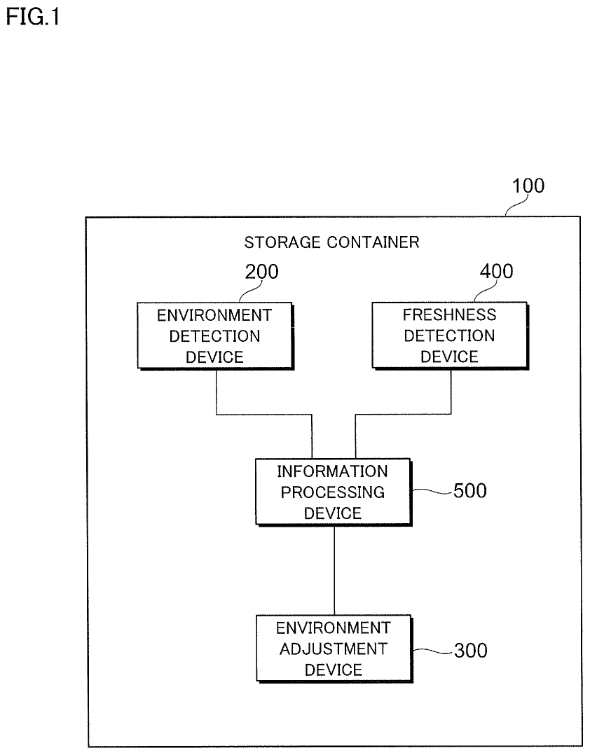

FIG. 1 is a diagram showing an overall configuration of a perishable product control system to which the present embodiment is applied. The perishable product control system of the present embodiment includes a storage container 100, an environment detection device 200, an environment adjustment device 300, a freshness detection device 400, and an information processing device 500.

The storage container 100 is a device that contains and stores perishable products. The storage container 100 has a delivery entrance (not shown) for carrying in the perishable products to be contained. By closing the delivery entrance of the storage container 100, inside space is sealed and separated from the external environment. The inside of the storage container 100 may be divided into multiple rooms. The storage container 100 is a container used for, for example, transporting the perishable products. As the storage container 100, those containing the perishable products in a refrigerated environment, those containing the perishable products in a frozen environment, those containing the perishable products in an ordinary-temperature environment, and so on can be assumed. The storage container 100 is an example of a containing device.

The environment detection device 200 is provided inside the storage container 100 and detects the environment inside the storage container 100 (the inside environment). The environment detection device 200 acquires data indicating the state of the internal space of the storage container 100 (hereinafter referred to as “environmental data”). Specifically, data indicating temperature, humidity, air pressure, the component of gas filling the internal space, etc., is acquired. Consequently, as the environment detection device 200, a temperature sensor, a humidity sensor, an air pressure sensor, sensors for various kinds of gas components, etc., can be used. In the case where the inside of the storage container 100 is divided into multiple rooms, the environmental data may be acquired individually from each room. The environment detection device 200 is an example of an environmental information acquisition unit.

The environment adjustment device 300 is provided in the storage container 100 and controls the inside environment of the storage container 100. Factors of the inside environment to be controlled by the environment adjustment device 300 correspond to factors of the inside environment to be detected by the environment detection device 200. Consequently. The environment adjustment device 300 controls the temperature, humidity, air pressure, components of gas filling the inside space, etc., to adjust the inside environment. In the case where the inside of the storage container 100 is divided into the multiple rooms, a different environment may be set for each room. The environment adjustment device 300 is an example of an adjustment unit.

The freshness detection device 400 detects the freshness of perishable products contained in the storage container 100. The freshness of a perishable product can be evaluated with various indexes. Specifically, “appearance (color, gloss),” “scent,” “taste,” “physical properties (hardness),” “water content,” “ingredients (sugar, acid),” etc. are used as the freshness indexes. In addition, for meat and fish, the index called “K value” regarding the ratio of ingredients contained in these perishable products is generally used as an index representing the freshness. Consequently, as the freshness detection device 400, in accordance with the kinds of perishable products to be contained, a freshness sensor capable of acquiring data regarding these various indexes (hereinafter, referred to as “freshness data”) is used.

The information processing device 500 processes data acquired by the environment detection device 200 and the freshness detection device 400, and controls the operation of the environment adjustment device 300 based on the obtained results. The information processing device 500 is implemented by, for example, a personal computer, a smartphone, an embedded computer in the storage container 100, etc. The information processing device 500 may be provided integrally with the storage container 100, or may be provided separately from the storage container 100 to acquire data from the environment detection device 200 and the freshness detection device 400 using a communication line. The configuration example in FIG. 1 shows the information processing device 500 provided integrally with the storage container 100. In the case where the information processing device 500 is provided separately from the storage container 100, the communication line for sending and receiving data may be a wired line or a wireless network.

### <Configuration of Information Processing Device 500>

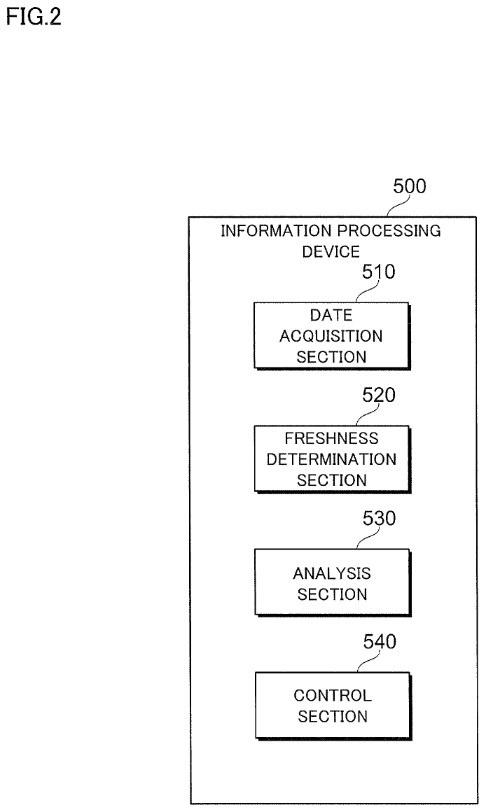

FIG. 2 is a diagram showing a functional configuration example of the information processing device 500. The information processing device 500 includes a data acquisition section 510, a freshness determination section 520, an analysis section 530, and a control section 540.

The data acquisition section 510 acquires data from the environment detection device 200 and the freshness detection device 400. Data is acquired, for example, at regular intervals. More specifically, environmental data is transmitted from the environment detection device 200 at regular intervals, which is acquired by the data acquisition section 510 of the information processing device 500. Freshness data is transmitted from the freshness detection device 400 at regular intervals, which is acquired by the data acquisition section 510 of the information processing device 500. The environmental data acquired from the environment detection device 200 is transmitted to the analysis section 530. The freshness data acquired from the freshness detection device 400 is transmitted to the freshness determination section 520.

The freshness determination section 520 determines the freshness of the perishable products contained in the storage container 100 based on the freshness data obtained by the freshness detection device 400. This provides information regarding the freshness of the perishable products. As described above, the freshness of the perishable products is determined based on the various freshness indexes. Therefore, the freshness determination section 520 determines the freshness by a method specified in accordance with the kind of perishable products contained in the storage container 100 and the type of freshness data obtained by the freshness detection device 400. The freshness may be determined using existing determination methods, for example, a method using the K values performed for fish and shellfish, or meat. The freshness determination section 520 is an example of a freshness information acquisition unit. In addition, the freshness detection device 400 and the freshness determination section 520 of the information processing device 500 are an example of a freshness measurement unit.

The analysis section 530 analyzes the information regarding the inside environment (the environmental data) acquired by the data acquisition section 510 and the freshness information of the perishable products determined by the freshness determination section 520, to thereby find the proper inside environment. The proper inside environment differs depending on the kinds and states of the perishable products contained in the storage container 100. It is needless to say that the proper inside environment differs depending on the kind of perishable product, but the proper inside environment is sometimes different for even the same kind of perishable product depending on the state thereof. For example, regarding tomatoes, the proper ranges of temperature and humidity are different between the fully ripened tomatoes and mature-green tomatoes. In addition, the fully ripened tomatoes produce a large amount of ethylene and have low ethylene sensitivity, whereas the mature-green tomatoes produce a small amount of ethylene and have high ethylene sensitivity. Therefore, the proper gas components are also different. In addition, the range of proper temperature for potatoes differs between the unripened state and the fully ripened state. Further, different freshness of the same kind of perishable product corresponds to different proper inside environment. For example, the state of high freshness, as compared to the state of low freshness, makes it possible to select the inside environment with the assumption of long-term storage. As described above, the analysis section 530 is required to identify the inside environment in consideration of various factors of the target perishable product.

In the present embodiment, the analysis section 530 uses a learning model obtained by the reinforcement learning to select the inside environment to be adjusted by the environment adjustment device 300. The learning model used by the analysis section 530 is obtained by performing reinforcement learning with settings of “state s” for the information regarding the inside environment and the freshness information, “action a” for the control condition of the environment adjustment device 300, and “reward r” for the degree of decrease in the freshness after elapse of a certain period of time. The “reward r” is set so that the smaller the decrease in the freshness, the larger the value of the “reward r.” Thus, according to the learning model, based on the “state s” obtained by the environment detection device 200, the freshness detection device 400, and the freshness determination section 520, the “action a” is optimized to minimize the decrease in the freshness (to maximize the “reward r”) after elapse of a certain period of time in the inside environment controlled by the “action a.”

In addition, the analysis section 530 may actually control the environment adjustment device 300 to perform freshness control of the perishable products while adjusting the inside environment by the information processing device 500 provided in the storage container 100, to thereby proceed with the reinforcement learning using the results of the freshness control. Specifically, the above-described learning model may be updated (setting of the “reward r” may be changed) based on the information regarding the inside environment actually adjusted, and the state of decrease in the freshness of the perishable product after elapse of a certain period of time from the start of control by the environment adjustment device 300. The analysis section 530 is an example of a learning unit and a reward decision unit.

The control section 540 controls the operation of the environment adjustment device 300 by generating a control instruction for the environment adjustment device 300, and transmitting the generated control instruction to the environment adjustment device 300. The control section 540 controls the environment adjustment device 300 to have the inside environment selected by the analysis section 530. In other words, the control section 540 controls the environment adjustment device 300 so that the environmental information acquired by the environment detection device 200 is that of the inside environment for the freshness of the perishable product learned at the analysis section 530 (the inside environment selected by the learning model). The control of the inside environment is individually performed for each of environmental factors, such as temperature, humidity, air pressure, and gas component, by comparing the current inside environment with the inside environment selected by the analysis section 530. Focusing on the inside temperature as an example, if the current inside temperature is the same as the inside temperature selected by the analysis section 530, the control section 540 controls the environment adjustment device 300 to maintain the current inside temperature. In addition, in the case where the current inside temperature is different from the inside temperature selected by the analysis section 530, the control section 540 controls the environment adjustment device 300 so that the inside temperature becomes the latter temperature.

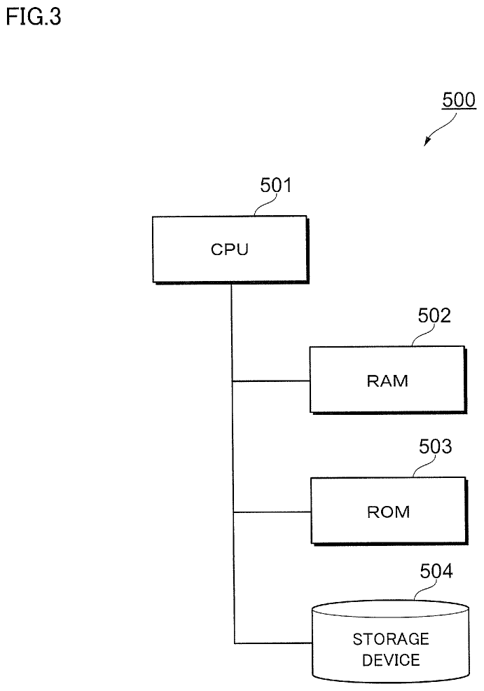

FIG. 3 is a diagram showing a hardware configuration example of the information processing device 500. The information processing device 500 is implemented by a computer. The computer that implements the information processing device 500 includes a CPU (Central Processing Unit) 501, which is an arithmetic unit, a RAM (Random Access Memory) 502, which is a storage unit, a ROM (Read Only Memory) 503, and a storage device 504. The RAM 502 is a main storage device (main memory), and used as a working memory when the CPU 501 performs arithmetic processing. The ROM 503 holds programs and data of setting values prepared in advance, etc., and the CPU 501 can execute processing by directly reading the programs and data from the ROM 503. The storage device 504 is a storing unit for programs and data. The storage device 504 stores the programs, and the CPU 501 reads the programs stored in the storage device 504 into the main storage device to execute thereof. In addition, the storage device 504 stores the processing results by the CPU 501 to preserve thereof. Moreover, the storage device 504 stores the learning model by the above-described reinforcement learning, which is used to select the inside environment. As the storage device 504, for example, a magnetic disk device, an SSD (Solid State Drive), etc. can be used.

In the case where the information processing device 500 is implemented by the computer shown in FIG. 3, each of the functions of the data acquisition section 510, the freshness determination section 420, the analysis section 530, and the control section 540, which have been described with reference to FIG. 2, is implemented by the CPU 501 executing the programs. The information processing device 500 implementing each of the above-described functions by execution of the programs by the CPU 501 is an example of a learning device.

### <Learning Example of Inside Environment Adjustment>

As described above, the analysis section 530 of the information processing device 500 uses a learning model obtained by the reinforcement learning to select the inside environment to be adjusted by the environment adjustment device 300. It has been described that, as the learning model, the model obtained by performing reinforcement learning with settings of “state s” for the information regarding the inside environment and the freshness information, “action a” for the control condition of the inside environment, and “reward r” for the degree of decrease in the freshness after elapse of a certain period of time is used; the further description will be given of the “state s,” the “action a,” and the “reward r.”

The inside environment is adjusted by the environment adjustment device 300 over a certain period of time (for example, 6 hours, 12 hours, 1 day, 3 days, 1 week, etc.). Consequently, for the inside environment as the “state s,” the inside environment during the certain period of time is considered. The operation of the environment adjustment device 300 during the certain period of time is the target of the “action a.” The “reward r” is determined based on the difference between the freshness at the start point and the freshness at the end point of the certain period of time.

The inside environment during the certain period of time will be considered further. If the inside environment is adjusted by the environment adjustment device 300 over a certain period of time, depending on the adjustment, the inside environment sometimes differs between the start point and the end point of the certain period of time. In addition, the operation of the environment adjustment device 300 during the certain period of time dynamically changes in some cases. In this case, even though the inside environment at the start point is the same, it is assumed that the degree of decrease in the freshness of the perishable product is different in response to the inside environment at the end point or midway through the period of time. Consequently, the “state s” is identified by considering the inside environment, not only at the start point, but also at the end point of and midway through the certain period of time. In addition, with regard to the control of the environment adjustment device 300 as the “action a,” even though the inside environment at the start point is the same, multiple “action a” can be set with the inside environments at the end point of and midway through the certain period of time that are different. Hereinafter, specific description will be given of some examples. In the following examples, the inside temperature will be focused on as a specific example of the inside environment.

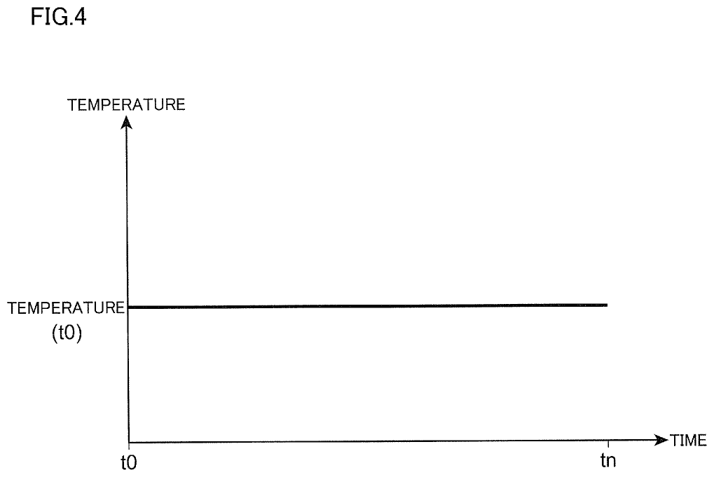

FIG. 4 is a diagram showing an example of the inside environment (inside temperature) over a certain period of time adjusted by the environment adjustment device 300. In the example shown in FIG. 4, the environment adjustment device 300 adjusts the inside temperature to be constant at the temperature (t0) during the period t(0-n) from the start point t0 to the end point tn. In this case, since the inside temperature is constant throughout the period t(0-n), the “state s” in the reinforcement learning is the temperature (t0). In addition, the operation of the environment adjustment device 300 as the “action a” is controlled so that the “state s” at the temperature (t0) continues for a certain period of time t(0-n). Then, the “reward r” is set based on the difference between the freshness of the perishable product at the point t0 and the freshness of the perishable product at the point tn.

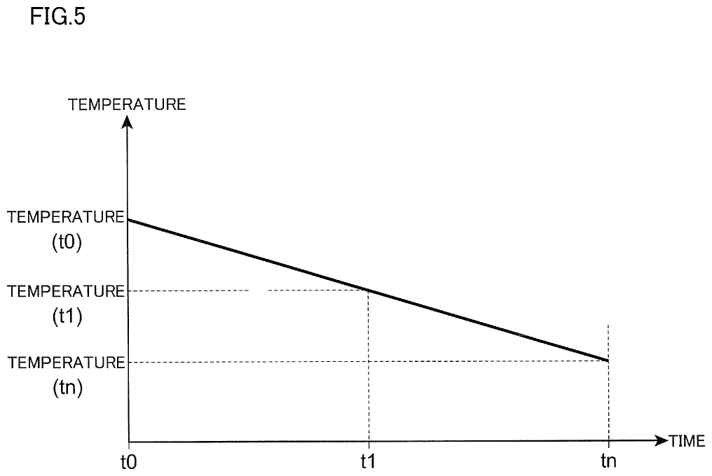

FIG. 5 is a diagram showing another example of the inside environment (inside temperature) over a certain period of time adjusted by the environment adjustment device 300. In the example shown in FIG. 5, the environment adjustment device 300 adjusts the inside temperature to change from the temperature (t0) to the temperature (tn) during the period t(0-n) from the start point t0 to the end point tn. Note that, in the example, the temperature change is adjusted to be constant with the elapsed time. In this case, the temperature (t0) at the start point t0 of the period of time t(0-n) and the temperature (tn) at the end point tn are different; accordingly, the temperature (t0) at the start point cannot simply be the “state s.” Therefore, the temperature at a specific point within the period t(0-n) is set to the “state s.” For example, it can be considered that a temperature (t1) at a middle point t1 between the point t0 and the point tn is set to the “state s.” This means that the “action a” is considered as controlling the operation of the environment adjustment device 300 so that the inside temperature at the point t0 is the temperature (t0), and the inside temperature after elapse of the period of time t(0-n) reaches the temperature (tn), and the “state s” is considered as the inside temperature (t1) throughout the period of time t(0-n). Then, the “reward r” is set based on the difference in the freshness between the point t0 and the point tn. Here, in the example shown in FIG. 5, since the degree of temperature change is constant,

period t(0-1)=period t(0-n)/2,

and

temperature (t1)=temperature (t0)+(temperature (tn)−temperature (t0))/2

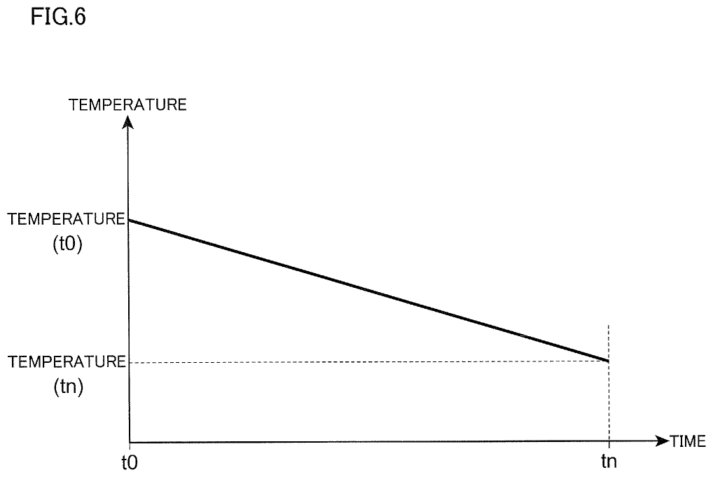

FIG. 6 is a diagram showing still another example of the inside environment (inside temperature) over a certain period of time adjusted by the environment adjustment device 300. In the example shown in FIG. 6, the environment adjustment device 300 adjusts the inside temperature to change from the temperature (t0) to the temperature (tn) during the period t(0-n) from the start point t0 to the end point tn. Note that, in the example, the temperature change is adjusted to be constant with the elapsed time. In this case, the temperature (t0) at the start point t0 of the period of time t(0-n) and the temperature (tn) at the end point tn are different; accordingly, the temperature (t0) at the start point cannot simply be the “state s.” The above is similar to the example described with reference to FIG. 5; however, in this example, both the temperature (t0) at the start point t0 and the temperature (tn) at the end point tn of the period t(0-n) are set as the “state s.” The “action a” is the operation control of the environment adjustment device 300, which implements such temperature changes. Then, the “reward r” is set based on the difference in the freshness between the point t0 and the point tn.

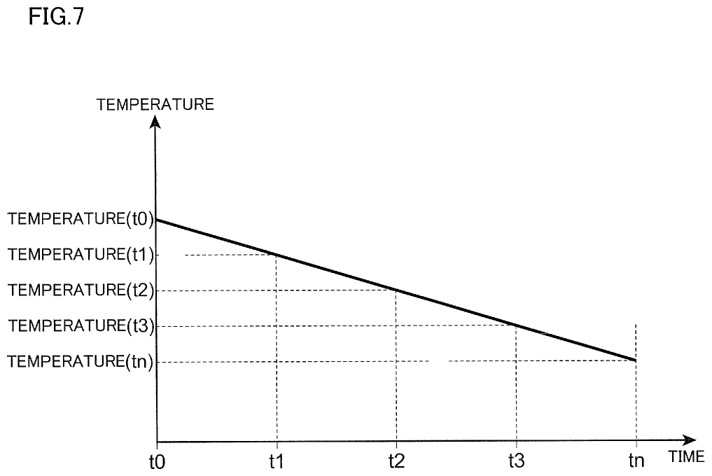

FIG. 7 is a diagram showing still another example of the inside environment (inside temperature) over a certain period of time adjusted by the environment adjustment device 300. In the example shown in FIG. 7, the environment adjustment device 300 adjusts the inside temperature to change from the temperature (t0) to the temperature (tn) during the period t(0-n) from the start point t0 to the end point tn. Note that, in FIG. 7, the temperature change is constant with the elapsed time, but this example does not specify how the temperature changes. For example, the temperature may be changed rapidly during the first half of the period t(0-n), and may be changed slowly during the second half. In addition, the temperature may be changed several times in a stepwise manner. In the example shown in FIG. 7, one or more points are set within the period t(0-n), and the “state s” is set based on the temperature at each point. For example, a representative value resulting from the statistical treatment of the temperature at each point may be set to the “state s.” An average value, a median value, etc. can be selected as the representative value, but the representative value may be set in accordance with how the temperature changes during the period t(0-n).

In the example shown in FIG. 7, three points, from the point t1 to the point t3, are set between the start point t0 and the end point tn, to thereby obtain five temperatures, the temperature (t0), the temperature (t1), the temperature (t2), the temperature (t3), and the temperature (tn). These points and temperatures may be set in accordance with how the temperature changes during the period of time t(0-n). Then, the representative value (for example, the average value) of these five temperatures is given as the “state s.” In addition, the operation control of the environment adjustment device 300 to achieve the above-described temperature change during the period t(0-n) is given as the “action a.” Then, the “reward r” is set based on the difference in the freshness between the point t0 and the point tn. Note that, in this example, the environment adjustment device 300 starts the control from the current inside environment to obtain the inside environment at the point when the period t(0-n) has elapsed (the point tn); accordingly, the point to obtain the inside environment may surely include the point tn.

Note that, in the examples described with reference to FIGS. 4 to 7, the description has been given of the case in which the inside temperature is controlled as one of the inside environments, but it is possible to similarly analyze each factor of the inside environment, such as humidity or gas component, that can be detected by the environment detection device 200 and adjusted by the environment adjustment device 300. In addition, each factor of the inside environment may be weighted, or conditions may be added in accordance with the kind and state of the perishable products, the freshness of the perishable products at the time of analysis, etc. For example, since the storable period in the storage container 100 differs depending on the types of perishable products, different periods can be set to the period t(0-n) shown in the above-described analysis example.

So far, the embodiment has been described, but the technical scope of the present disclosure is not limited to the above-described embodiment. For example, in the above-described embodiment, the information processing device 500 has been described as the device that implements each of the function to process the data acquired by the environment detection device 200 and the freshness detection device 400, the function to control the operation of the environment adjustment device 300 based on the results of the processing, and the function to perform the reinforcement learning based on the acquired data; however, these functions may be implemented by individual pieces of hardware. The device for performing the learning function is also implemented as a learning device that learns by use of data acquired and collected by the environment detection devices 200 and the freshness detection devices 400 of the multiple storage containers 100.

In addition, the above-described embodiment has shown that the analysis section 530 of the information processing device 500 learns to obtain a proper inside environment in accordance with the kinds and states of the perishable products. Consequently, when the freshness of the perishable products is controlled, the user identifies the perishable products to be controlled on the information processing device 500, and uses the learning model corresponding to the identified perishable products to control the freshness. In contrast thereto, a means of identifying the kinds of perishable products subjected to the freshness control may be provided in the information processing device 500. This makes it possible for the information processing device 500 to identify the perishable products to be controlled and perform the freshness control using the corresponding learning model when the freshness of the perishable products is controlled. The perishable products may be identified, for example, by image analysis using images obtained by photographing the perishable products. Other changes and configuration alternatives that do not deviate from the scope of the technical principles of the present disclosure are included in the present disclosure.

Here, the above-described embodiment can be viewed as follows. The learning device of the present disclosure includes: a freshness determination section 520 acquiring information regarding freshness of a perishable product contained in a storage container; and an analysis section 530 learning an inside environment of the storage container for the freshness of the perishable product acquired by the freshness determination section 520 to decide a reward used in the learning. The analysis section 530 is a learning device of the inside environment for perishable products that decides the reward based on the decrease in the freshness over a certain period of time under the inside environment for the freshness determined based on the freshness acquired by the freshness determination section 520, and learns the inside environment for the freshness based on the decided reward.

In this manner, the reinforcement learning can be performed on control conditions of the perishable product environment by using the information regarding the freshness of perishable products to automatically control the perishable product environment.

Here, in the case where the inside environments before and after the certain period of time are constant, the analysis section 530 may decide the reward based on the inside environment at the start point of the certain period of time.

In this manner, the environmental control to provide the constant inside environment over a certain period of time can be learned.

Alternatively, in the case where the inside environment before and after the certain period of time is different, the analysis section 530 may decide the reward based on the inside environment at a specific point during the certain period of time.

In this manner, in the case where the inside environment changes during the certain period of time, the environmental control based on the inside environment at the specific point can be learned.

Alternatively, in the case where the inside environment before and after the certain period of time is different, the analysis section 530 may decide the reward based on the inside environment at the start point and the inside environment at the end point of the certain period of time.

In this manner, in the case where the inside environment changes during the certain period of time, the environmental control based on the inside environments at the start point and the end point of the certain period of time can be learned.

Alternatively, in the case where the inside environments before and after the certain period of time are different, the analysis section 530 may find the representative value of environmental information values indicating the inside environment at multiple points during the certain period of time, and may decide the reward based on the representative value.

In this manner, the environmental control in response to the change in the inside environment over a certain period of time can be learned.

In addition, the above-described embodiment can also be viewed as follows. The containing device of the present disclosure includes: the storage container 100 containing perishable products; the environment adjustment device 300 adjusting the inside environment including at least the temperature inside the storage container 100; the environment detection device 200 acquiring the environmental information including at least the temperature inside the storage container 100; the freshness detection device 400 and the freshness determination section 520 measuring the freshness of the perishable products contained in the storage container 100; and the analysis section 530 learning the inside environment of the storage container 100 for the freshness of the perishable products measured by the freshness detection device 400 and the freshness determination section 520 to decide the reward. Based on the freshness measured by the freshness detection device 400 and the freshness determination section 520, the environmental information acquired by the environment detection device 200, and the learning results of the analysis section 530, the environment adjustment device 300 operates to cause the inside environment of the storage container 100 to serve as the inside environment for the freshness. The analysis section 530 is the perishable product containing device that decides the reward based on the decrease in the freshness over a certain period of time under the inside environment adjusted by the environment adjustment device 300, and learns the inside environment for the freshness based on the decided reward.

In this manner, the reinforcement learning can be performed on control conditions of the inside environment by using the information regarding the freshness of perishable products obtained from the storage container 100 to automatically control the perishable product environment.

Here, the analysis section 530 may learn the inside environment for the freshness of perishable products measured by the freshness detection device 400 and the freshness determination section 520 to maximize the reward decided by the analysis section 530, and the environment adjustment device 300 may adjust the inside environment of the storage container 100 to cause the environmental information acquired by the environment detection device 200 to serve as the inside environment for the freshness learned by the analysis section 530.

In this manner, it is possible to learn the control of the inside environment optimized with respect to decrease in the freshness of perishable products.

In addition, in the case where the inside environments before and after the certain period of time are different, the analysis section 530 may decide the reward based on the inside environment at one or more points during the certain period of time.

In this manner, the environmental control in response to the change in the inside environment over a certain period of time can be learned.

Moreover, the inside environments at one or more points during the certain period of time may include the inside environment at the end point of the certain period of time.

In this manner, it is possible to learn the environmental control assuming the inside environment when the certain period of time has elapsed.

Moreover, the program of the present disclosure causes a computer to implement the function of acquiring the information regarding the freshness of perishable products contained in the storage container, the function of learning the inside environment of the storage container for the acquired freshness of perishable products, and the function of deciding the reward used in the learning, and, in the function of deciding the reward, the reward is decided based on the decrease in the freshness over the certain period of time under the inside environment for the freshness, the inside environment being determined based on the freshness of the perishable product acquired by the function of acquiring the information regarding the freshness, and in the function of learning, the inside environment for the freshness is learned based on the reward decided by the function of deciding the reward.

According to the computer installing the program, the reinforcement learning can be performed on the control conditions of the perishable product environment by using the information regarding the freshness of perishable products to automatically control the perishable product environment.

### REFERENCE SIGNS LIST

100 Storage container
200 Environment detection device
300 Environment adjustment device
400 Freshness detection device
500 Information processing device
510 Data acquisition section
520 Freshness determination section
530 Analysis section
540 Control section

1. A learning device of an inside environment of a storage container for a perishable product, the device comprising:
a freshness information acquisition unit acquiring information regarding freshness of a perishable product contained in the storage container;
a learning unit learning the inside environment of the storage container for the freshness of the perishable product acquired by the freshness information acquisition unit; and
a reward decision unit deciding a reward used by the learning unit, wherein
the reward decision unit decides the reward based on a decrease in the freshness over a certain period of time under the inside environment for the freshness determined based on the freshness acquired by the freshness information acquisition unit, and
the learning unit learns the inside environment for the freshness based on the reward decided by the reward decision unit.

2. The learning device of an inside environment of a storage container for a perishable product according to claim 1, wherein, when inside environments before and after the certain period of time are constant, the reward decision unit decides the reward based on an inside environment at a start point of the certain period of time.

3. The learning device of an inside environment of a storage container for a perishable product according to claim 1, wherein, when inside environments before and after the certain period of time are different, the reward decision unit decides the reward based on an inside environment at a specific point during the certain period of time.

4. The learning device of an inside environment of a storage container for a perishable product according to claim 1, wherein, when inside environments before and after the certain period of time are different, the reward decision unit decides the reward based on an inside environment at a start point and an inside environment at an end point of the certain period of time.

5. The learning device of an inside environment of a storage container for a perishable product according to claim 1, wherein, when inside environments before and after the certain period of time are different, the reward decision unit finds a representative value of environmental information values indicating inside environments at multiple points during the certain period of time, and decides the reward based on the representative value.

6. A perishable product containing device comprising:
a storage container containing a perishable product;
an adjustment unit adjusting an inside environment of the storage container, the inside environment including at least a temperature;
an environmental information acquisition unit acquiring environmental information inside the container, the environmental information including at least the temperature;
a freshness measurement unit measuring freshness of the perishable product contained in the storage container;
a learning unit learning the inside environment of the storage container for the freshness of the perishable product measured by the freshness measurement unit; and
a reward decision unit deciding a reward used by the learning unit, wherein,
based on the freshness measured by the freshness measurement unit, the environmental information acquired by the environmental information acquisition unit, and a learning result of the learning unit, the adjustment unit operates to cause the inside environment of the storage container to serve as an inside environment for the freshness,
the reward decision unit decides the reward based on a decrease in the freshness over a certain period of time under the inside environment adjusted by the adjustment unit, and
the learning unit learns the inside environment for the freshness based on the reward decided by the reward decision unit.

7. The perishable product containing device according to claim 6, wherein
the learning unit learns the inside environment for the freshness of the perishable product measured by the freshness measurement unit to maximize the reward decided by the reward decision unit, and
the adjustment unit adjusts the inside environment of the storage container to cause the environmental information acquired by the environmental information acquisition unit to serve as the inside environment for the freshness learned by the learning unit.

8. The perishable product containing device according to claim 7, wherein, when inside environments before and after the certain period of time are different, the reward decision unit decides the reward based on an inside environment at one or more points during the certain period of time.

9. The perishable product containing device according to claim 8, wherein the inside environment at one or more points during the certain period of time includes an inside environment at an end point of the certain period of time.

10. A program causing a computer to implement:
a function of acquiring information regarding freshness of a perishable product contained in a storage container;
a function of learning an inside environment of the storage container for the acquired freshness of the perishable product; and
a function of deciding a reward used in the learning, wherein,
in the function of deciding the reward, the reward is decided based on a decrease in the freshness over a certain period of time under the inside environment for the freshness, the inside environment being determined based on the freshness of the perishable product acquired by the function of acquiring the information regarding the freshness, and,
in the function of learning, the inside environment for the freshness is learned based on the reward decided by the function of deciding the reward.
